(19) United States
(12) Patent Application Publication
(10) Pub. No.: US 2005/0129783 A1
(43) Pub. Date: Jun. 16, 2005

(54) Composition and method for treatment of neurophysiological conditions and maintenance of neurophysiological health

(76) Inventors: Edward Larry McCleary, Incline Village, NV (US); Carl A. Forest, Boulder, CO (US); Christine McCleary, Incline Village, NV (US)

Correspondence Address: PATTON BOGGS, 1660 LINCOLN ST, SUITE 2050, DENVER, CO, 80264, US

(21) Appl. No.: 10/986,924

(22) Filed: Nov. 12, 2004

Related U.S. Application Data
(63) Continuation-in-part of application No. 09/837,562, filed on Apr. 19, 2001.
(63) Continuation-in-part of application No. 10/890,067, filed on Jul. 12, 2004.
Application No. 10/890,067 is a continuation-in-part of application No. 10/462,958, filed on Jun. 17, 2003.
Application No. 10/890,067 is a continuation-in-part of application No. 10/616,674, filed on Jul. 10, 2003.
(60) Provisional application No. 60/520,466, filed on Nov. 14, 2003.
(60) Provisional application No. 60/536,286, filed on Jan. 13, 2004.

(51) Int. Cl. A61K 35/78; A61K 33/26; A61K 31/56; A61K 31/522; A61K 31/51

(52) U.S. Cl. 424/646; 424/733; 424/741; 424/769; 424/771; 424/777; 424/764; 514/548; 514/356; 514/285; 514/291; 514/276; 514/263.31; 514/350; 514/171; 514/561; 514/567; 514/553

(57) ABSTRACT

A composition and method for therapeutic treatment of undesirable neurophysiologic conditions, such as those associated with menopause, the peri-menstrual period, mental conditions such as mood disorders, hyperactivity, impulsiveness, difficulty concentrating, chronic pain conditions, and including neurodegenerative disorders such as age-associated or acoustic-related hearing loss, central, peripheral or autonomic neuropathies and for maintenance of neurophysiologic health, the composition comprising an effective amount of: three agents selected from the group consisting of vinpocetine, huperzine A, magnesium, theanine, pyro-glutamic acid, dextromethorphan, and Vitex agnus-castus fruit; one agent selected from the group consisting of thiamin, niacin, pyridoxine, vanadium, and acetaminophen; and three agents selected from the group consisting of valerian spp, passiflora spp, magnolia spp, scutellaria spp, taurine, melatonin, GABA and black cohosh. The composition may also include an effective amount of an agent selected from the group consisting of white willow bark, dehydroepiandrosterone (DHEA), pregnenolone, ketone bodies, 5-hydroxytryptophan (5-HTP) and a racemic mixture of D,L phenylalanine (DLPA). The method comprises administering the above composition to a human for a therapeutically effective period. The composition may also be combined with a food, beverage, condiment, spice or salad dressing base to provide a food, beverage, condiment, spice or salad dressing product designed to overcome or prevent undesirable conditions frequently associated with menopause, chronic pain or neurophysiologic degeneration.

### RELATED APPLICATIONS

This application is a continuation-in-part of U.S. patent application Ser. No. 09/837,562 filed Apr. 19, 2001 and is also a continuation-in-part of U.S. patent application Ser. No. 10/890,067 filed Jul. 12, 2004, which is itself a continuation-in-part of U.S. patent application Ser. No. 10/462,958 filed on Jun. 17, 2003 and is also a continuation-in-part of U.S. patent application Ser. No. 10/616,674 filed Jul. 10, 2003. Through the above-mentioned application Ser. No. 10/890,067, this application also claims the benefit of U.S. Provisional Patent Application No. 60/520,466 filed Nov. 14, 2003 and U.S. Provisional Patent Application Ser. No. 60/536,286 filed Jan. 13, 2004. All of the patent applications are hereby incorporated by reference to the same extent as though fully disclosed herein.

### BACKGROUND OF THE INVENTION

1. Field of the Invention

This invention relates in general to nutraceuticals, including dietary supplements, nutritional supplements, medical foods, botanical drugs and drugs, and in particular neutraceuticals and methods of use for therapeutic treatment undesirable neurophysiologic conditions, such as those associated with menopause, the peri-menstrual period, mental conditions such as mood disorders, hyperactivity, impulsiveness, difficulty concentrating, chronic pain conditions, and including neurodegenerative disorders such as age-associated or acoustic-related hearing loss, central, peripheral or autonomic neuropathies, prophylactically for avoiding such conditions, or for maintenance of neurophysiologic health. In Another aspect, the invention also relates to the combination of such nutraceuticals with foods, beverages, spices, condiments and salad dressings.

2. Statement of the Problem

Over the last century, life span has increased substantially, yet the age of the menopausal transition has remained relatively stable at about 50 years. Because of this, an increasing number of women are living a larger fraction of their lives in the post-menopausal state. Menopause and peri-menopause are frequently associated with numerous symptoms that affect the quality of life. These include, but are not limited to, pain/discomfort, bloating, breast tenderness, memory, concentration and cognitive symptoms, mood disorders, depression, anxiety, hot flashes/night sweats, sleep disturbances and others. These symptoms may also occur independently of menopause or the menstrual cycle and are still to be included in this patent application. In addition, men may suffer from similar symptoms for related reasons and these are included as well. Current therapeutic approaches such as hormonal replacement therapy (HRT), while providing symptomatic relief, may increase the risk of various hormonally sensitive cancers or may not be well-tolerated for one reason or another by large numbers of women. “Natural” treatments such as the administration of plant phyto-estrogens may alleviate some symptoms, yet they are believed to function via up-regulation of estrogenic signaling pathways and thus may put at risk women who have a predisposition for the development of hormonally responsive tumors. Hence, there is a need for a safe, effective, non-hormonal therapeutic approach to these difficult problems.

A clinically common group of frequently disabling painful/neurogenic disorders is that of migraine and atypical migraine syndromes, reflex sympathetic dystrophy, and other neurogenic, traumatic, degenerative, inflammatory or metabolic neural conditions. Recent theories suggest the central etiologic role played by alterations in the interaction and regulation of excitatory/inhibitory neurotransmitters. Pharmaceutical modulation of these pathways has been effective in controlling these syndromes. However, the pharmaceutical modulators all have undesirable side effects. Hence, there is a need for a safe, effective, non-pharmaceutical treatment of such pain disorders.

It would also be highly desirable to have such nutraceuticals available in a manner that made them transparently easy to use in the course of everyday life, and at a time and quantity that was more compatible with human metabolism.

### SUMMARY OF THE INVENTION

The invention solves the above problems with a composition and method that effectively modulates neurotransmitters in the nervous system, i.e., the brain, spinal cord, and peripheral nervous system.

For many years it was believed that menopause occurred simply due to exhaustion of the remaining ovarian follicles. The approach of this disclosure is that this is an incomplete explanation of a much more complicated process involving the central nervous system, the ovary, and a complex time-dependent interactive process culminating in the development of the frequently prolonged peri-menopausal period and subsequent transition into the acyclic menopausal state. Herein, the resultant symptomatology is viewed much like a drug withdrawal syndrome wherein the drug has modified the brain by long term exposure, withdrawal, and the production of ‘release’ type responses. The view herein is that the appropriate approach involves an investigation of the relevant neurophysiologic alterations. Based on this approach, a novel composition has been formulated based upon the nervous system reaction to the alteration in the gonadal hormonal environment. This composition may be used therapeutically, or simply for the maintenance of healthy conditions.

The compositions disclosed herein modulate neurotransmission in the nervous system, influencing both central (brain and spinal cord) and peripheral (peripheral nerve) neurotransmission. They predominantly influence the major excitatory (glutamate) and inhibitory (GABA) neurotransmitters and modify the excitatory/inhibitory neurotransmitter balance.

In researching the application of neurotransmitter modulation to menopausal and peri-menopausal symptomatology, it was discovered that the medial literature relating to neurotransmission modulation demonstrates the beneficial role of “atypical” drugs in the control of various types of pain syndromes. For example, Gabapentin has been shown to play a key role in neurogenic facial and spinal pain disorders. In a similar fashion, Tegretol, Dilantin, Valproic Acid and Amitriptyline have demonstrated efficacy in numerous, difficult to treat, painful conditions such as trigeminal neuralgia. One explanation for these successful interventions may be that these drugs affect various excitatory and inhibitory neurotransmitters throughout the neuraxis. Thus, it was postulated that the neurotransmission modulators useful for menopausal and peri-menopausal symptom relief may also have beneficial effects for pain disorders. Initial studies indicate that this is correct.

The invention provides a composition for therapeutic treatment of undesirable neurophysiologic conditions, such as those associated with menopause, the peri-menstrual period, mental conditions such as mood disorders, hyperactivity, impulsiveness, difficulty concentrating, chronic pain conditions, and including neurodegenerative disorders such as age-associated or acoustic-related hearing loss, central, peripheral or autonomic neuropathies, for avoiding such conditions, or for maintenance of neurophysiologic health,; the composition comprising an effective amount of: three agents selected from the group consisting of vinpocetine, huperzine A, magnesium, theanine, pyro-glutamic acid, dextromethorphan, and Vitex agnus-castus fruit; one agent selected from the group consisting of thiamin, niacin, pyridoxine, vanadium, and acetaminophen; and three agents selected from the group consisting of valerian spp, passiflora spp, magnolia spp, scutellaria spp, taurine, melatonin, GABA (gamma amino butyric acid) and black cohosh. Preferably, the composition further includes an effective amount of an agent selected from the group consisting of white willow bark, dehydroepiandrosterone (DHEA), pregnenolone, ketone bodies, 5-hydroxytryptophan (5-HTP) and a racemic mixture of D,L phenylalanine (DLPA). Preferably, the ketone bodies comprise: β-hydroxybutyrate and acetoacetate, or their salts/precursors; and/or medium chain triglycerides (MCT) or their C6 to C14 fatty acid analogs or precursors. Preferably, the composition comprises magnesium, vinpocetine, huperzine, theanine, pyridoxine, valerian spp, taurine, GABA and magnolia spp. Preferably, the composition also includes 5HTP and white willow bark, or alternatively, melatonin, and Vitex agnus-castus fruit. Preferably, the composition may also include medium chain triglycerides. In an alternative embodiment, the composition comprises vinpocetine, huperzine, magnesium, dextromethorphan, acetaminophen, taurine, black cohosh, and magnolia spp. In a further embodiment, the composition comprises magnesium, theanine, pyro-glutamic acid, thiamin, valerian spp, passiflora spp, magnolia spp, GABA and white willow bark. In a further embodiment, the composition comprises magnesium, vinpocetine, huperzine, thiamin, niacin, pyridoxine, taurine, magnolia spp, melatonin, and MCT oil. In another embodiment, the composition comprises theanine, magnesium, pyro-glutamic acid, thiamin, valerian spp, passiflora spp, GABA, magnolia spp, and DLPA. In yet another embodiment, the composition comprises magnesium, Vitex agnus-castus fruit, acetaminophen, black cohosh, valerian spp, magnolia spp, melatonin, and 5 HTP.

The invention also provides a method for therapeutic treatment of undesirable neurophysiologic conditions, such as those associated with menopause, the peri-menstrual period, mental conditions such as mood disorders, hyperactivity, impulsiveness, difficulty concentrating, chronic pain conditions, and including neurodegenerative disorders such as age-associated or acoustic-related hearing loss, central, peripheral or autonomic neuropathies, for avoiding such conditions, or for maintenance of neurophysiologic health, the method comprising orally or parenterally administering to the human for an effective period, an effective amount of a composition comprising: three agents selected from the group consisting of vinpocetine, huperzine A, magnesium, theanine, pyro-glutamic acid, dextromethorphan, and Vitex agnus-castus fruit; one agent selected from the group consisting of thiamin, niacin, pyridoxine, vanadium, and acetaminophen; and three agents selected from the group consisting of valerian spp, passiflora spp, magnolia spp, scutellaria spp, taurine, melatonin, GABA and black cohosh. Preferably, the composition administered further includes an agent selected from the group consisting of white willow bark, dehydroepiandrosterone (DHEA), pregnenolone, ketone bodies, 5-hydroxytryptophan (5-HTP) and a racemic mixture of D,L phenylalanine (DLPA). Preferably, the ketone bodies comprise: β-hydroxybutyrate and acetoacetate, or their salts/precursors; and/or medium chain triglycerides or their C6 to C14 fatty acid analogs or precursors.

The invention also provides a solution to the above problem by systematically incorporating the above compositions into foods, condiments and spices, salad dressings and beverages, preferably into certain foods that have become an everyday part of the modern diet. In the more preferred embodiments, the compositions are incorporated into foods that are normally eaten by persons having the chronic problems that the nutraceuticals address. In the most preferred embodiment, details of the metabolic process are used to select combinations of a food, a condiment, a spice, a beverage, or a salad dressing with nutraceuticals that will most likely address particular chronic dietary, nutritional or health problems mentioned above as well as other chronic health issues.

The invention provides a food useful for mitigating undesirable neurophysiologic conditions, such as those associated with menopause, the peri-menstrual period, mental conditions such as mood disorders, hyperactivity, impulsiveness, difficulty concentrating, chronic pain conditions, and including neurodegenerative disorders such as age-associated or acoustic-related hearing loss, central, peripheral or autonomic neuropathies, for avoiding such conditions, or for maintenance of neurophysiologic health, the food comprising: a food base; the above-described composition, which composition is present in an amount in excess of the amount of the composition present naturally in foods. In other aspects, a beverage base, a condiment or spice base, or a salad dressing base can be substituted for the food base, to provide a beverage, condiment of spice, or salad dressing useful for mitigating undesirable neurophysiologic conditions, such as those associated with menopause, the peri-menstrual period, mental conditions such as mood disorders, hyperactivity, impulsiveness, difficulty concentrating, chronic pain conditions, and including neurodegenerative disorders such as age-associated or acoustic-related hearing loss, central, peripheral or autonomic neuropathies, for avoiding such conditions, or for maintenance of neurophysiologic health.

The invention provides a food, beverage, condiment, spice or salad dressing product designed for neurophysiologic health, the product comprising: a food, beverage, condiment, spice or salad dressing base; and a nutraceutical comprising an effective amount of: three agents selected from the group consisting of vinpocetine, huperzine A, magnesium, theanine, pyro-glutamic acid, dextromethorphan, and Vitex agnus-castus fruit; one agent selected from the group consisting of thiamin, niacin, pyridoxine, vanadium, and acetaminophen; and three agents selected from the group consisting of valerian spp, passiflora spp, magnolia spp, scutellaria spp, taurine, melatonin, GABA and black cohosh; the nutraceutical present in an amount in excess of the amount of the nutraceutical naturally present in the food, beverage, condiment, spice or salad dressing base. In one aspect, the base is a beverage base and the beverage is an alcoholic beverage. In another aspect, the base is a food base and the food is preferably selected from the group consisting of: vegetables and vegetable food products such as baked beans, tomato-based products like tomato paste, stewed tomatoes, spaghetti sauce, pizza sauce, vegetable oils, fruit and fruit food products such as jellies, jams and syrups, cereals, trail mix, cookies, pasta, flours including wheat, soy, oat, and potato flour, whey, chocolate, candy, tofu, bagels, baked goods such as bread, cakes, pizza dough and mixes for these, pancakes and waffles and mixes for these, soups, trail mix, nutritional bars, peanut butter, potato chips, corn chips, crackers, meats, and meat food products, such as lunch meats, hot dogs and sausage, milk food products, such as ice cream, yogurt, cheese and butter. In a further aspect, the base is a beverage base and the beverage is preferably selected from the group consisting of: soft drinks, tea, coffee, milk, fruit juices, and sports drinks. In yet another aspect, the base is a condiment or spice base and the condiment or spice is preferably selected from the group consisting of: steak sauce, mustard, catsup, soy sauce, chili sauce, chip dips, salsa, pickles, horseradish, curry, chili powder, salt, pepper, vinegar, cinnamon, garlic and garlic powder, garlic oil, onion and onion powder and onion oil. Preferably, the nutraceutical further includes an effective amount of an agent selected from the group consisting of white willow bark, dehydroepiandrosterone (DHEA), pregnenolone, ketone bodies, 5-hydroxytryptophan (5-HTP) and a racemic mixture of D,L phenylalanine (DLPA). Preferably, the ketone bodies comprise: β-hydroxybutyrate and acetoacetate, or their salts/precursor; and medium chain triglycerides or their C6 to C14 fatty acid analogs or precursors. Preferably, the nutraceutical comprises magnesium, vinpocetine, huperzine, theanine, pyridoxine, valerian spp, taurine, and magnolia spp. Preferably, the nutraceutical further comprises 5HTP and white willow bark. In another embodiment, the nutraceutical further comprises DHEA, melatonin, and Vitex agnus-castus fruit. In a further embodiment, the nutraceutical further comprises medium chain triglycerides. In yet another embodiment, the nutraceutical preferably, comprises vinpocetine, huperzine, magnesium, dextromethorphan, acetaminophen, taurine, black cohosh, and magnolia spp. In still another embodiment, the nutraceutical comprises magnesium, theanine, pyro-glutamic acid, thiamin, valerian spp, passiflora spp, magnolia spp, and white willow bark. In still a further embodiment, the nutraceutical comprises magnesium, vinpocetine, huperzine, thiamin, niacin, pyridoxine, taurine, magnolia spp, melatonin, and MCT oil. In a further embodiment, the nutraceutical comprises theanine, magnesium, pyro-glutamic acid, thiamin, valerian spp, passiflora spp, magnolia spp, and DLPA. In yet and additional embodiment, the nutraceutical comprises magnesium, Vitex agnus-castus fruit, acetaminophen, black cohosh, valerian spp, magnolia spp, melatonin, and 5 HTP.

The invention provides “nutraceutical” as opposed to pharmaceutical approaches for conditions that have up to now been very difficult to treat clinically. Since all the agents of the inventive compound are ones that humans have been exposed to for hundreds of thousands of years without significant ill effects, and since the compounds merely enhance or retard natural processes associated with the neurosystem, the compounds of the invention overall should be safer than the pharmaceuticals mentioned above. Numerous other features and advantages of the invention will become apparent upon reading the following detailed description of the invention.

### DETAILED DESCRIPTION OF THE INVENTION

1. Overview

The invention relates to certain compositions which are of benefit to human well being. In general, we shall label these compositions as “nutraceuticals” herein. Depending the point of view of the persons using them, nutraceuticals may be called, dietary supplements, nutritional supplements, botanical drugs, medical foods, and sometimes even drugs. In the future, other terms may be applied to these compounds. The invention is intended to cover these compositions when used for the purposes stated, no matter what they are called.

As discussed above, the current therapeutic approach to symptoms associated with peri-menopause involves administration of potent steroidal hormones such as estrogenic and/or progestational compounds. “Natural” and non-therapeutic approaches frequently involve administration of plant based estrogenic compounds such as phytoestrogens. These all produce their biological action by binding to hormone receptors. The pharmaceutical approach is referred to as HRT and, while frequently effective in symptomatic control, is associated with cancer risk and other occasionally fatal side effects such as liver necrosis. The current disclosure teaches a non-HRT approach to a similar range of conditions, but acts via modulation of neuro-transmitter activity as described herein.

Recently attention has been paid to the possibility that age-related changes in the central nervous system, specifically the hypothalamus, play pivotal roles in the events leading to menopause and the symptoms associated with these changes. Observations suggest that desynchronization of neural cycles accelerates the depletion of ovarian follicles. This is consistent with the observation that well before the depletion of ovarian follicles fertility decreases markedly, reproductive cycles become increasingly irregular in length, and patterns of pituitary gland gonadotropin secretion are altered.

As subtle, early, cyclical changes in hypothalamic neuronal function develop, ovarian response becomes altered. Estrogen profiles are modified, as is ovarian feedback received by the brain. Hypertrophy of neurons has been described in the infundibular nucleus of the hypothalamus in post-menopausal women. This may be reversed by estrogen which further illustrates the close relationship between the ovary and the brain.

Precise changes in neuro-chemical and neuro-endocrine signals that dictate the evolving pattern of gonadotropin secretion during this transitional period are being identified. They are responsible for subsequent changes in ovarian follicular function. Coalescence of subtle hypothalamic changes taking place during this process contributes to the onset of irregular cycles with subsequent progression over time to the acyclic state with its associated symptomatology. Identification, and characterization, of the functional changes in the nervous system ultimately responsible for these clinical symptoms provide the insight for the formulation of a new therapeutic approach free of the toxicity of the current HRT regimens.

Analysis of the hormone withdrawal state induced in the brain by ovarian involution, and comparison with conceptually similar drug withdrawal states, provide a picture of the brain in a state where it is released from the control of gonadal hormones and related neuro-steroids. This produces central nervous system excitation analogous to the elevated sympathetic nervous system activity seen with narcotic withdrawal syndromes. Similar results would be expected with down-regulation of endogenous opiate neurotransmission. Manifestations at the cellular level include selective neuronal proliferation associated with up-regulation of gene expression and protein synthesis. These changes may be suppressed by the exogenous administration of estrogenic compounds. Not only are the molecular changes reversed, but the clinical phenotype is reverted as well.

These changes illustrate the profound interaction between brain and ovarian function. During the peri-menopausal period, the nervous system alters the neurotransmitter balance in the hypothalamus. This involves one of the main excitatory neurotransmitters, glutamic acid, and the inhibitory amino acid neurotransmitter gamma aminobutyric acid (GABA). The glutamate-GABA balance regulates the synthesis and secretion of gonadotropin releasing hormones (GnRH) from the hypothalamus and their release into the hypothalamic-pituitary portal vascular system.

By understanding the mechanisms of action of various herbs, minerals and other nutritional supplements as well as their impact upon the germane neuro-chemical pathways, the nutraceuticals may be combined in novel combinations and provided for administration using various delivery systems allowing for safe treatment for a variety of hormonally driven symptoms including hot flashes, sweats, mood alterations, pain syndromes, cognitive alterations, and related peri-menstrual and peri-partum symptoms. That peri-menstrual symptoms are amenable to such manipulations is suggested by the association of symptomatology across the menstrual cycle with alterations in amino acid neurotransmission.

There is growing evidence that GnRH activity is intimately related to the development of many of the hormonally related symptoms that develop in the peri-menopausal state. For example, GnRH gene expression is elevated in the medial basal hypothalamus (MBH) of post-menopausal women. This elevation is associated with neuronal hyperactivity. In animal studies using direct neuronal monitoring techniques, volleys of multi-unit electrical activity have been recorded in the MBH. These represent the neural correlate of the GnRH pulse generator. Long term ovariectomy in monkeys increases the duration and frequency of these volleys. Estrogen administration decreases them. These observations reveal the intricate relationship between brain electrical activity and GnRH synthesis and secretion. Of primary interest is the fact that control of the central regulation of opiate and GnRH activity may be produced by direct neurotransmitter manipulation, or by exposure to estrogen. This observation forms the basis for the development of non-hormonal therapies for hormonally related disorders.

GnRH therefore represents a critical neuro-endocrine link in the production of symptoms seen in the peri-menopausal state. If non-hormonal therapies are developed that down-regulate the nervous system hyperactivity and associated over production of GnRH based upon modulation of the glutamate-GABA imbalance, these would constitute safer solutions to a range of common problems that have a significant functional impact. Such therapeutic approaches would be free of the toxicity of current HRT regimens. The current disclosure proposes a formulation based upon this approach. It involves the selection and administration of a combination of agents chosen based upon their ability to beneficially modulate the neurochemistry outlined above in specific clinical situations. Based upon this disclosure, those skilled in the field can guide the selection and amount of the various agents listed as the active ingredients herein taking into account the clinical situation.

As indicated above, there is evidence that a state of excessive nervous system activation is the basis for the constellation of symptomatology associated with the peri-menopausal state. This excessive nervous system activation is a result of the withdrawal of long-standing hormonal-nervous tissue interaction. The neurological correlates of the long term ovarian-brain interaction are manifested as structural and functional synaptic alterations. As the hormonal milieu changes, these molecular alterations in the brain react to the release of hormonal control. It is submitted that this interaction forms the basis for the symptoms seen in the peri-menopausal transition. These include hot flashes, sweats, mood alterations, neuro-psychiatric phenomenology, cognitive and behavioral changes, pain syndromes, as well as symptoms related to hormonal changes seen peri-menstrually and during and after pregnancy.

The hyperactive brain state influences pituitary gonadotropin secretion. This is due, in large part, to alterations in GnRH secretion in the hypothalamus. Such changes represent aberrations in hypothalamic electrical pulse generation; the basic neuronal process that directs the complex neuro-endocrine axis. Evidence suggests that deterioration in the coupling of neurotransmitter signals responsible for regulation of GnRH secretion causes the initial changes in the altered pattern of gonadotropin secretion. The primary feature of these initial neuro-chemical changes is that daily rhythmicity is affected far more than overall activity level. This finding that the rhythmicity, or cycling, of many transmitter systems changes during aging has led to the hypothesis that deterioration of the “biological clock”, or its ability to communicate with GnRH secreting neurons, alters gonadotropin secretion.

GnRH plays important roles in regulation of the menstrual cycle. The neurons responsible for GnRH synthesis also project to other intracranial regions raising the possibility that GnRH has actions not directly related to reproduction. Hot flashes, for example, are episodes of sudden vasodilation in the facial and upper abdominal regions designed to facilitate heat loss. Peripheral concentrations of GnRH are elevated in relation to these episodes. Because hot flashes occur even after hypophysectomy (removal of the pituitary gland), the conclusion is that GnRH plays the critical role in their production. These episodes are considered malfunctions of thermoregulation. In the post-menopausal state it is believed that endogenous hypothalamic opiate neuronal activity falls. Exogenous provision of physiologic concentrations of estrogen or progesterone is able to prevent this fall in opiatergic tone, and subsequently prevent the hot flash symptom complex. In post-menopausal women, when concentrations of these hormones fall, a central noradrenergic excitation/hyperactivity ensues associated with falling endogenous opiate levels. This correlates with elevation of body core temperature preceding the hot flash and likely triggers the subsequent activation of the vaso-motor cooling process.

The basic mediators of neuronal hyperactivity and increased noradrenergic tone are voltage-gated calcium channels. When activated, they allow calcium ions to enter neurons. Down-regulation of calcium channel activity has been shown to inhibit norepinephrine release. Additionally, glutamate, the chief excitatory neurotransmitter in the nervous system, acts through G protein-coupled receptors which, when activated, also increase intra-cellular calcium levels. Hence, by blocking both the premonitory norepinephrine surge and the associated release of glutamate (and hence secretion of GnRH), a bi-modal approach exists designed to prevent the production of hot flashes and related symptomatology.

In rat studies, N-methyl-D-aspartate (NMDA) glutamate receptors produced a facilitatory effect upon GnRH pulsatility, while GABA A receptors mediated inhibitory effects. This glutamate-GABA duality forms the basis for GnRH neurotransmitter regulation. Importantly, this modulation may be achieved without the risks associated with HRT. On the contrary, several of the agents proposed herein manifest anti-estrogenic activity and may beneficially modulate risk for the development of hormonally active tumors such as those that arise in breast ductal tissue or endometrium.

A major adverse effect associated with hormone treatment for prostate cancer is hot flashes. The reduction of circulating testosterone, or its ability to bind to receptors, represents the most likely mechanism for the production of hot flashes in men. This is analogous to the fall in estrogen levels in women and produces a similar response in the brain. The approach advocated in this application applies to this situation as well. Women having symptoms related to the administration of anti-estrogen therapy would also be candidates for the therapy recommended herein.

2. Description of the Compounds

The primary agents and doses or serving sizes designed to beneficially modulate GnRH secretion are listed below.

| | | |
|---|---|---|
| | | PREFERRED |
| | DAILY DOSE OR | DAILY DOSE |
| NAME | SERVING SIZE | OR SERVING SIZE |

I Anti-Excitatory Agents

| | | |
|---|---|---|
| A) | | |
| Vinpocetine | 3 mg to 100 mg | 12 mg to 50 mg |
| Huperzine A | 5 mcg to 500 mcg | 20 mcg to 300 mcg |
| Magnesium | 50 mg to 2000 mg | 200 mg to 1000 mg |
| Theanine | 50 mg to 1000 mg | 200 mg to 600 mg |
| Pyro-glutamic Acid | 5 mg to 500 mg | 50 mg to 200 mg |
| Dextromethorphan | 5 mg to 200 mg | 30 mg to 100 mg |
| Vitex agnus-castus fruit* | 2 mg to 40 mg | 4 mg to 20 mg |

*Standardized for casticin

| | | |
|---|---|---|
| B) | | |
| Thiamin | 0.1 mg to 100 mg | 10 mg to 50 mg |
| Niacin | 10 mg to 1000 mg | 20 mg to 500 mg |
| Pyridoxine | 0.1 mg to 100 mg | 20 mg to 75 m |
| Vanadium | 1 mcg to 10 mg | 10 mcg to 5 mg |
| Acetaminophen | 10 mg to 1000 mg | 20 mg to 300 mg |

II GABAergic Agents

| | | |
|---|---|---|
| Valerian spp | 50 mg to 1000 mg | 100 mg to 500 mg |
| Passiflora spp | 25 mg to 750 mg | 75 mg to 500 mg |
| Magnolia spp | 500 mg to 5000 mg | 700 mg to 3000 mg |
| Scutellaria spp | 30 mg to 650 mg | 50 mg to 500 mg |
| Taurine | 50 mg to 1500 mg | 100 mg to 750 mg |
| Melatonin | 0.1 mg to 20 mg | 0.5 mg to 10 mg |
| Black Cohosh | 10 mg to 200 mg | 20 mg to 100 mg |
| GABA | 1 mg to 2000 mg | 25 mg to 1000 mg |

III Anti-Pyretics

| | | |
|---|---|---|
| White Willow Bark* | 10 mg to 1000 mg | 100 mg to 500 mg |

*The given dosages or serving sizes are the amounts of salicylates
in the white willow bark.

IV Neuro-Steroids

| | | |
|---|---|---|
| DHEA | 5 mg to 300 mg | 25 mg to 150 mg |
| (Dehydro- | | |
| epiandrosterone) | | |
| Pregnenolone | 5 mg to 300 mg | 25 mg to 150 mg |

V Ketogenic Agents (or precursors)

| | | |
|---|---|---|
| Ketone Bodies | 5 g to 300 g | 10 g to 100 g |

Ketone bodies include: β-hydroxybutyrate, acetoacetate or their salts/precursors; and medium chain triglycerides (MCT) or their fatty acid analogues or metabolic precursors. Medium chain triglycerides are triglycerides having fatty acids with structural backbones that contain 6 to 14 carbon atoms. Typical MCTs are octanoic acid, decanoic acid, and hexanoic acid. Generally, commercially available MCT is an oil including a mixture of several or all of the MCTs.

| | | |
|---|---|---|
| | | PREFERRED |
| | DAILY DOSE OR | DAILY DOSE |
| NAME | SERVING SIZE | OR SERVING SIZE |

VI Serotoninergic Agents

| | | |
|---|---|---|
| 5-Hydroxytryptophan (5- | 10 mg to 1000 mg | 50 mg to 600 mg |
| HTP) | | |

VII Enkefalinase Inhibitors

| | | |
|---|---|---|
| DLPA | 10 mg to 10 g | 50 mg to 2 g |

DLPA is a racemic mixture of D, L phenylalanine.

Mixtures of these agents are preferred to address the particular neurotransmitter abnormalities that exist in each particular case. Choices from Groups I and II are recommended as the basis for a useful formula. For a broader approach, agents from Groups III and VI may be added. Neuro-steroidal agents from Group IV may then be added, as indicated, and for other cases Group V agents may be utilized. The discussion and the examples below will enable those skilled in the art to make appropriate combinations.

Oral formulations taken in divided doses or servings on a daily basis, or as needed, are preferred. Sublingual and trans-dermal vehicles may also be used.

These agents and combinations thereof modulate neurotransmission in the nervous system. This involves both neurotransmission in the central (brain and spinal cord) and peripheral (peripheral nerve) neurotransmission. The agents predominantly influence the major excitatory (glutamate) and inhibitory (GABA) neurotransmitters and modify the excitatory/inhibitory neurotransmitter balance. Primarily, via pharmaceutical manipulation, the beneficial role of “atypical” drugs has been demonstrated in the control of various types of pain syndromes. Gabapentin has been shown to play a key role in neurogenic facial and spinal pain disorders. In a similar fashion, Tegretol, Dilantin, Valproic Acid and Amitriptyline have demonstrated efficacy in numerous, difficult to treat painful conditions such as trigeminal neuralgia. One explanation for these successful interventions is related to the effects of the drugs upon various excitatory and inhibitory neurotransmitters throughout the neuraxis. Similar actions are observed upon administration of the nutraceutical agents, and combinations of these agents, described in this disclosure.

A clinically common group of frequently disabling painful disorders is that of migraine and atypical migraine syndromes. Recent theories suggest the central etiologic role played by alterations in the interaction and regulation of excitatory/inhibitory neurotransmitters. Pharmaceutical modulation of these pathways has been effective in controlling these syndromes. Such benefits are obtained by similar modulation of these pathways by the nutraceutical agents listed here.

A further category of pain syndrome is that of facial, lingual, optic, otic, ophthalmic or pharyngeal pain. These are often attributed a neurogenic etiology and would be expected to be amenable to the various therapies discussed herein. In addition, painful and/or degenerative central and peripheral neuropathies caused by metabolic, traumatic, inflammatory, or age-related etiologies are therapeutic targets.

Many women, especially at the menopausal transition or in conjunction with the menstrual cycle, manifest unusual abdominal, or pelvic, pain symptoms that are difficult to diagnose, categorize and treat. Based upon similar reasoning that these may be related to abnormalities in the visceral nerve fibers innervating the enteric, reproductive, or urinary structures these pain symptoms should exhibit a beneficial response to the neurotransmitter modulation described herein.

The racemic mixture of D,L phenylalanine (DLPA) is included in the agents listed above to enhance the efficacy of the proposed combinations for pain control. DLPA, while rarely effective as a stand-alone agent when used for pain control, acts synergistically due to its ability to inhibit the enzyme enkefalinase, an endogenous opiate degrading enzyme. The net effect is to up-regulate naturally occurring pain relieving compounds without the associated addiction risk. Similar actions of DPLA may be anticipated in the central nervous system and would tend to up-regulate opiatergic neurotransmission.

3. Guidelines For Formulation Of Various Combinations Of Ingredients

In this discussion, the assumption is made that the person doing the formulating is someone skilled in the field who understands the goals to be achieved by any specific formulation based upon:
A) The unique needs of the individual(s) for whom the formulation is directed;
B) Guidance from the teachings herein;
C) A detailed understanding of the biochemical, physiologic and medical properties of each of the proposed ingredients, other than the properties discussed herein, and how they interact with each other, which understanding is available via a thorough knowledge of the medical literature;
D) A detailed understanding of the condition to be treated, and any other drugs or nutraceuticals the individual is consuming to avoid adverse response;
E) The physician's desired goals for the patient, and/or the patient's goals;
F) Past clinical experience;
G) The thought process of each physician or health practitioner used to formulate a specific and unique health care plan from an almost infinite number of possibilities for each patient.

Preferred general combinations of the listed active agents are discussed below, and following the general discussion, specific combinations for specific conditions are recommended. These preferred and recommended combinations are based on agent actions that are known in the literature. The literature relied on in creating these combinations fills several filing cabinets, and thus to include it all, or even a significant portion of it, herein would make this disclosure unwieldy and would bury the important teachings in a mass of information. However, the following goals have driven the formulations:
1) to capitalize upon multiple synergistic actions;
2) to achieve the desired clinical outcome with minimal toxicity;
3) to produce a product with a reasonable cost profile;
4) to maximize product efficacy by agent choices that incorporate and utilize agent actions that provide enhanced activity for each specific condition;
5) to formulate products via a holistic approach, as contrasted to the traditional pharmaceutical “magic bullet” approach, based upon the interactions of the currently-known pathways responsible for the production of any specific condition.

As discussed above, agents from Groups I and II form the functional basis for all formulations. Choices are clinically driven as outlined previously. This involves the individual agent choices, amounts of each agent, number of agents chosen, ratios of the various ingredients, and choices from each specific group. Based upon the above general principles, any formulation should include at least three agents from group IA, at least one agent from Group IB, and at least three agents from Group II. Depending upon specific requirements additional agents are chosen from Groups III, IV, V, VI, and VII. Examples are listed below to illustrate these points. In the examples, the amounts given are a daily dose or servings, which can be administered in one to several portions.

### EXAMPLE 1

A simple formulation for menopausal women with a wide, but fairly typical, symptom profile including hot flash symptoms, night sweats, intermittent irritability, and mild memory concerns includes the following combination of agents.

| | | |
|---|---|---|
| Magnesium | 400 | mg |
| Vinpocetine | 2 | mg |
| Huperzine | 50 | mcg |
| Theanine | 100 | mg |
| Pyridoxine | 20 | mg |
| Valerian spp | 100 | mg |
| Taurine | 150 | mg |
| Magnolia spp | 700 | mg |

### EXAMPLE 2

A related combination that would target menopausal women with migraine symptoms would include the agents listed in Example 1 with the addition of:

| | |
|---|---|
| 5 HTP | 250 mg |
| White Willow Bark | 400 mg |
| Additional Magnesium | 300 mg |

### EXAMPLE 3

A sample preparation for women with more severe menopausal symptomatology would include the agents in Example 1 with the addition of the following:

| | | |
|---|---|---|
| DHEA | 25 | mg |
| Melatonin | 2 | mcg |
| Vitex agnus-castus fruit | 10 | mg |
| (mg casticin) | | |

### EXAMPLE 4

A formulation helpful for symptomatic menopausal women who gain weight would include the agents in Example 1 plus:
MCT oil 10 cc.

### EXAMPLE 5

Symptomatic women who might not tolerate the mild sedation and/or confusion related to the ingredients listed in Example 1 should have a better response with the following combination:

| | | |
|---|---|---|
| Vinpocetine | 5 | mg |
| Huperzine | 150 | mcg |
| Magnesium | 300 | mg |
| Dextromethorphan | 30 | mg |
| Acetaminophen | 300 | mg |
| Taurine | 500 | mg |
| Black Cohosh | 10 | mg |
| Magnolia spp | 300 | mg |

### EXAMPLE 6

A formulation that should be considered for migraine headaches would include:

| | |
|---|---|
| Magnesium | 1000 mg |
| Theanine | 200 mg |
| Pyro-glutamic acid | 25 mg |
| Thiamin | 20 mg |
| Valerian spp | 100 mg |
| Passiflora spp | 75 mg |
| Magnolia spp | 600 mg |
| White Willow Bark | 650 mg |

### EXAMPLE 7

A combination designed for a patient with painful diabetic peripheral neuropathy would include:

| | | |
|---|---|---|
| Magnesium | 600 | mg |
| Vinpocetine | 2 | mg |
| Huperzine | 50 | mcg |
| Thiamin | 20 | mg |
| Niacin | 25 | mg |
| Pyridoxine | 20 | mg |
| Taurine | 500 | mg |
| Magnolia spp | 500 | mg |
| Melatonin | 1 | mg |
| MCT oil | 5 | cc |

### EXAMPLE 8

A formulation for a patient with low back pain related to nerve root compression secondary to spinal stenosis would be:

| | |
|---|---|
| Theanine | 250 mg |
| Magnesium | 250 mg |
| Pyro-glutamic acid | 50 mg |
| Thiamin | 20 mg |
| Valerian spp | 100 mg |
| Passiflora spp | 50 mg |
| Magnolia spp | 500 mg |
| DLPA | 500 mg |

### EXAMPLE 9

A formulation for a 25 year old female with severe pre-menstrual symptomatology would include:

| | |
|---|---|
| Magnesium | 1000 mg |
| Theanine | 100 mg |
| Vitex agnus-castus fruit (mg | 25 mg |
| casticin) | |
| Acetaminophen | 325 mg |
| Black Cohosh | 40 mg |
| Valerian spp | 250 mg |
| Magnolia spp | 750 mg |
| Melatonin | 3 mg |
| 5 HTP | 300 mg |

### EXAMPLE 10

A Formulation for a human suffering from age-related
hearing loss would include:

| | | |
|---|---|---|
| Magnesium | 800 | mg |
| Huperzine A | 100 | mcg |
| Theanine | 75 | mg |
| Vinpocetine | 5 | mg |
| Pyro-glutamic acid | 25 | mg |
| Thiamin | 10 | mg |
| Niacin | 20 | mg |
| Pyridoxine | 15 | mg |
| Taurine | 200 | mg |
| GABA | 200 | mg |
| Passiflora spp | 100 | mg |
| Valerian spp | 100 | mg |

It may also include:

| | | |
|---|---|---|
| α Lipoic Acid | 150 | mg |
| Trimethylglycine | 200 | mg |
| Phosphatidyl choline | 125 | mg |
| L-carnitine | 100 | mg |
| Acetyl-L-carnitine | 125 | mg |
| Folic acid | 400 | mcg |

A feature of the invention is that the compositions provide “nutraceutical” as opposed to pharmaceutical approaches for these taxing clinical conditions. A related feature of this approach is that the compounds of the invention overall should be safer than the pharmaceuticals mentioned above. Further benefits include a lower risk of addiction and improved cost profile.

The term “effective period” with respect to the administration of the composition in the method of this invention means that period of time sufficient to modulate neurotransmission in the human. Preferably, the composition of this invention is administered on a daily basis for a period of at least three weeks, more preferably at least six weeks.

With respect to the amounts of the individual components of the composition of this invention, the term “effective amount” means that amount of the component which, when used in combination with the other components in the composition, will provide the composition with the capability of modulating neurotransmitter function so as to alleviate menopausal symptoms or chronic pain.

4. Use in Foods, Beverages, Condiments, Spices and Salad Dressings

As described in detail in U.S. patent application Ser. No. 10/890,067, which is hereby incorporated by reference to the same extent as fully disclosed herein, the above-described compositions can also be administered by incorporating them into a food, beverage, condiment, spice or salad dressing.

In the preferred embodiment of the invention, the nutraceutical portion of the food, beverage, condiment, spice or salad dressing/nutraceutical combination, is not just any fortification thought to be essential or useful in reducing lipid storage, but comprises a combination of ingredients that have been clinically demonstrated, individually or together, to reduce lipid storage.

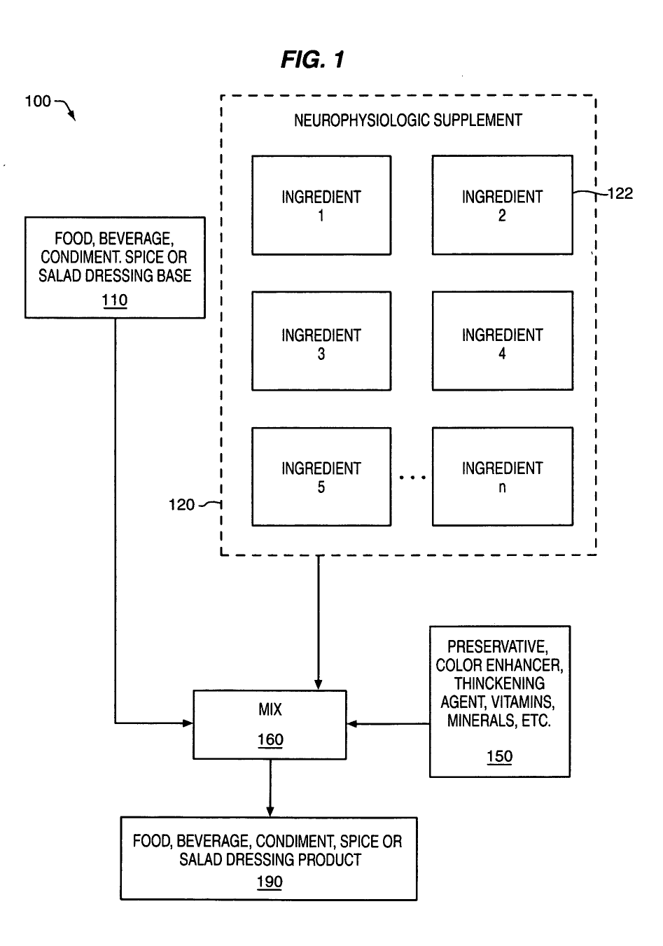

FIG. 1 illustrates the food, beverage, condiment or spice, or salad dressing product 190 according to the invention and the method of making it. The food, beverage, condiment or spice, or salad dressing with lipid reduction nutraceutical product includes a food, beverage, condiment or spice, or salad dressing base 110 and a neurophysiologic nutraceutical 120 as discussed above. Optionally, it may also include other non-nutraceutical ingredients 150, such as preservatives, color enhancers, thickening agents, vitamins, minerals, or other such additive ingredients. Food, beverage, condiment or spice, or salad dressing 110, neurophysiologic nutraceutical 120, and any other desirable ingredient 150 are mixed at 160 to create food, beverage, condiment or spice, or salad dressing product 190.

In the following, we will disclose a number of different food, beverage, condiment or spice, and salad dressing bases that have been found to be useful to make the food, beverage, condiment, spice or salad dressing product 190. However, it should be understood that these are exemplary, that is, only illustrative, and not intended to be exhaustive.

In this disclosure, all terms that relate to the food, beverage, condiment or spice, or salad dressing component have the meaning commonly used in the food. beverage, condiment, spice and salad dressing arts, respectively. “Food” is defined herein as “material consisting essentially of protein, carbohydrate, and fat used in the body of an organism to sustain growth, repair, and vital processes and to furnish energy. “Food” includes appetizers. Preferably, the word “food” herein is limited to foods as defined above in solid form, to distinguish foods from beverages, though it also includes some liquids that are commonly thought of as foods and not as beverages, such as vegetable oils, syrups, and soups. In this disclosure, “condiment” means something used to enhance the flavor of food; especially, a pungent seasoning. Spices include any of various aromatic vegetable products (as pepper, cinnamon, nutmeg, mace, allspice, ginger, cloves) used in cookery to season food and to flavor foods (as sauces, pickles, cakes) or combinations thereof. Condiments do not include salad dressing, since salad dressings are considered to be a separate category herein. In this disclosure “beverage” means a liquid for drinking, that is, a liquid that is typically drunk for the purpose of refreshment, and specifically does not include water, medicines, or liquid nutraceuticalary substances.

Similarly, all terms that relate to the nutraceutical component have the meaning they commonly have in the nutraceutical art. In general, referral to a specific ingredient of a nutraceutical is to be understood as referring to all forms of that ingredient. For example, the term “alpha lipoic acid” (ALA) or “lipoic acid” refers to ALA, lipoic acid, thioctic acid, R alpha lipoic acid, and racemic mixtures thereof. As another example, a salt is interchangeable with its corresponding acid, e.g. aspartate and aspartic acid are interchangeable. Similarly, when other ingredients may exist in various forms, a listing of one form refers to all the various forms, unless expressly noted otherwise.

The terms “food base”, “beverage base”, “condiment or spice base”, or “salad dressing base” as used herein are intended to include any and all low-fat, low-carbohydrate, or “lite” foods, beverages, condiments, spices and salad dressings also. For example, a low-fat or light salad dressing may be derived from any of the salad dressing bases given below by substituting water and a thickening agent, such as xanthan gum, for all or a portion of the oil given in the formulation, or by other substitutions and formulations known in the salad dressing art. The invention includes the combination of such low-fat foods with the specialized nutraceuticals according to the invention.

All of the nutraceuticals discussed herein are nutraceuticals that are known in the metabolic and medical literature, and thus the dosages or serving sizes that are useful are known in these arts. It is not possible herein to reproduce the vast quantity of literature that addresses dosages or serving sizes of particular nutraceuticals that have been found to be useful. However, we have provided exemplary dosages or serving sizes to provide illustrative examples. In all cases, the nutraceutical is present in an amount in excess of the amount of the nutraceutical present naturally in the particular food, beverage, condiment, spice, or salad dressing. In general, the amount of a nutraceutical to be added to a serving of food, beverage, condiment or salad dressing is the appropriate amount for a daily dosage or serving size. In other cases in which it is expected that a food, beverage, condiment, spice or salad dressing, or a combination of these, with the nutraceutical added are expected to be consumed several times a day, the amounts given are essentially halved—that is, it is assumed that the intake of the foods and/or beverages will be twice a day. It should be evident that if particular diets call for ingesting of particular foods, beverages, condiments, spices or salad dressings containing the nutraceuticals more than twice a day, then the amounts of ingredients in serving portion should be correspondingly reduced. If a food or beverage amount is such that it is intended for multiple servings, then the amount should also be correspondingly increased. Preferably, the foods, beverages, condiments or spices, and salad dressings are provided with a variety of different dosages or serving sizes that are fractions or multiples of the preferred daily dosage or serving size so that a consumer can chose to a dosage or serving size that is less or more than the preferred daily dosage or serving size as the consumer desires. As an example of how an actual product according to the invention can be varied, exemplary salad dressings are disclosed below. The amounts given for the exemplary salad dressings are the amounts sufficient to fill a small bottle of about twelve to fifteen ounces of weight loss salad dressing. It is intended that the amounts of the ingredients given in the various salad dressing bases should be mixed with the amounts of the ingredients given in the various weight loss nutraceuticals. The amounts are designed so that a serving of salad dressing is two ounces. It is assumed that salads are eaten twice a day so that two salads, or four ounces of salad dressing, provide effective daily amounts of weight loss ingredients. The various amounts given for salad dressing bases may be varied as known in the art of salad dressings, and the various amounts given for weight loss ingredients may be varied as known in the art of weight loss nutraceuticals. Similarly, for the other foods, beverages, condiments, spices and salad dressings. In the following, “tsp” means teaspoon, “tbsp” means tablespoon, and “mg” is milligrams.

4A. Food Bases

The food bases do not include chewing gum, inert ingredients by themselves, or other conventional materials for delivering health nutraceuticals. The common definition of food necessarily includes only materials that provide protein, fat, and/or carbohydrates. That is, as discussed above, the definition of food is intended to be narrow and is not to be broadened to include materials not commonly recognized as food.

The preferred foods are vegetables and vegetable food products such as baked beans, tomato-based products like tomato paste, stewed tomatoes, spaghetti sauce, pizza sauce, vegetable oils, fruit and fruit food products such as jellies, jams and syrups, cereals, trail mix, cookies, pasta, flours including wheat, soy, oat, and potato flour, whey, chocolate, candy, tofu, bagels, baked goods such as bread, cakes, pizza dough and mixes for these, pancakes and waffles and mixes for these, soups, trail mix, nutritional bars, peanut butter, potato chips, corn chips, crackers, meats, and meat food products, such as lunch meats, hot dogs and sausage, milk food products, such as ice cream, yogurt, cheese and butter.

4B. Beverage Bases

The term “beverage” is used in its common meaning which does not include water or medicines. Beverages are limited to those which are drunk for refreshment. We divide beverages into non-alcoholic and alcoholic beverages because these are related to very different industries and are thought about differently by those skilled in the art of nutraceuticals.

I. Non-alcoholic beverages: The preferred non-alcoholic beverages are soft drinks, tea, coffee, milk, fruit juices, and sports drinks. However, any non-alcoholic refreshment beverage is contemplated by the invention.

II. Alcoholic beverages: The preferred alcoholic beverages are beer, wine, liquors, liqueurs, flavored alcohols, brandies, cocktails, aperitifs, and cordials. However, any alcoholic refreshment beverage is contemplated by the invention.

4C. Condiment and Spices Bases

Condiments include anything commonly referred to as a condiment, including steak sauce, mustard, catsup, soy sauce, chili sauce, chip dips, salsa, pickles, horseradish, curry, chili powder, etc.

Spices include anything commonly referred to as a spice including salt, pepper, vinegar, cinnamon, garlic and garlic powder or garlic oil, onion and onion powder or onion oil, etc.

3D. Salad Dressing Bases

Any salad dressing can be used as a base for the invention. We shall use salad dressing to provide detailed examples of the process and product of the invention. In these examples, when vegetable oil is mentioned, any vegetable oil may be used, preferably soybean oil or canola oil.

Table A. Oil and Vinegar Salad Dressing Base
½ cup water
½ cup vegetable oil
¼ cup vinegar (red wine vinegar may be used)
1 tsp sugar (optional)
½ tsp salt
½ tsp pepper

Small amounts of spices such as onion powder and/or garlic powder may be added.

Table B. Caesar Salad Dressing Base
1 egg, raw
3 tbsp lemon juice
garlic (about ½ teaspoon garlic powder or a large clove)
1 cup olive oil (other vegetable oil may be used)
¼ to ½ cup grated Parmesan or Romano cheese
1 tsp high fructose corn syrup
4 tbls anchovy paste
¼ tsp salt and pepper to taste

Table C. French Salad Dressing Base
½ cup vegetable oil
⅓ cup high fructose corn syrup
¼ cup water
3 tbls vinegar
½ tsp salt
½ tsp whey
⅓tsp modified food starch
pinch paprika
Enough yellow food color #5 and/or #6 to give desired color

Table D. Ranch Salad Dressing Base
½ cup water
½ cup vegetable oil
3 tbls vinegar
3 tbls sugar
1 egg yolk
2 tbls buttermilk
½ tsp salt
½ tsp whey
½ tsp modified food starch
¼ teaspoon malted dextrin

Table E. Bleu Cheese Salad Dressing Base
½ cup vegetable oil
¼ cup vinegar
¼ cup water
3 tbls blue cheese
2 tbls high fructose corn syrup
1 egg yolk
1 tsp lactic acid
   Other salad dressing bases include Russian dressing, Thousand Island dressing, Italian dressing, mayonnaise, balsamic vinaigrette, green goddess, and any other dressing used on salads.

From the above disclosure, and the nutraceutical examples given above, those skilled in the art of foods will understand the invention and how to implement it in a wide variety of foods, beverages, condiments, spices, and salad dressings.

There have been described novel compositions and processes for treatment of neurophysiologic conditions and maintenance of neurophysiologic health, in particular conditions associated with menopause and chronic pain. While the invention has been described in terms of specific embodiments, it should be understood that the particular embodiments described within this specification are for purposes of example and should not be construed to limit the invention which will be described in the claims below. Further, it is evident that those skilled in the art may now make numerous uses and modifications of the specific embodiments described, without departing from the inventive concepts. Consequently, the invention is to be construed as embracing each and every novel feature and novel combination of features present in and/or possessed by the compositions and methods described and by their equivalents.

1. A composition for therapeutic treatment of undesirable neurophysiologic conditions, such as those associated with menopause, the peri-menstrual period, mental conditions such as mood disorders, hyperactivity, impulsiveness, difficulty concentrating, chronic pain conditions, and including neurodegenerative disorders such as age-associated or acoustic-related hearing loss, central, peripheral or autonomic neuropathies, for avoiding such conditions, or for maintenance of neurophysiologic health, said composition comprising an effective amount of: three agents selected from the group consisting of vinpocetine, huperzine A, magnesium, theanine, pyro-glutamic acid, dextromethorphan, and Vitex agnus-castus fruit; one agent selected from the group consisting of thiamin, niacin, pyridoxine, vanadium, and acetaminophen; and three agents selected from the group consisting of valerian spp, passiflora spp, magnolia spp, scutellaria spp, taurine, melatonin, GABA and black cohosh.

2. A composition as in claim 1 and further including an effective amount of an agent selected from the group consisting of white willow bark, dehydroepiandrosterone (DHEA), pregnenolone, ketone bodies, 5-hydroxytryptophan (5-HTP) and a racemic mixture of D,L phenylalanine (DLPA).

3. A composition as in claim 2, wherein said ketone bodies comprise: β-hydroxybutyrate and acetoacetate, or their salts/precursor; and medium chain triglycerides or their C6 to C14 fatty acid analogs or precursors.

4. A composition as in claim 1 comprising magnesium, vinpocetine, huperzine, theanine, pyridoxine, valerian spp, taurine, and magnolia spp.

5. A composition as in claim 4 and further comprising 5HTP and white willow bark.

6. A composition as in claim 4 and further comprising DHEA, melatonin, and Vitex agnus-castus fruit.

7. A composition as in claim 4 and further comprising medium chain triglycerides.

8. A composition as in claim 1 comprising vinpocetine, huperzine, magnesium, dextromethorphan, acetaminophen, taurine, black cohosh, and magnolia spp.

9. A composition as in claim 1 comprising magnesium, theanine, pyro-glutamic acid, thiamin, valerian spp, passiflora spp, magnolia spp, and white willow bark.

10. A composition as in claim 1 comprising magnesium, vinpocetine, huperzine, thiamin, niacin, pyridoxine, taurine, magnolia spp, melatonin, and MCT oil.

11. A composition as in claim 1 comprising theanine, magnesium, pyro-glutamic acid, thiamin, valerian spp, passiflora spp, magnolia spp, and DLPA.

12. A composition as in claim 1 comprising magnesium, Vitex agnus-castus fruit, acetaminophen, black cohosh, valerian spp, magnolia spp, melatonin, and 5 HTP.

13. A method for therapeutic treatment of undesirable neurophysiologic conditions, such as those associated with menopause, the peri-menstrual period, mental conditions such as mood disorders, hyperactivity, impulsiveness, difficulty concentrating, chronic pain conditions, and including neurodegenerative disorders such as age-associated or acoustic-related hearing loss, central, peripheral or autonomic neuropathies, for avoiding such conditions, or for maintenance of neurophysiologic health, said method comprising orally or parenterally administering to the human for a therapeutically effective period, an effective amount of a composition comprising: three agents selected from the group consisting of vinpocetine, huperzine A, magnesium, theanine, pyro-glutamic acid, dextromethorphan, and Vitex agnus-castus fruit; one agent selected from the group consisting of thiamin, niacin, pyridoxine, vanadium, and acetaminophen; and three agents selected from the group consisting of valerian spp, passiflora spp, magnolia spp, scutellaria spp, taurine, melatonin, GABA and black cohosh.

14. A method as in claim 13 wherein said composition further includes an effective amount of an agent selected from the group consisting of white willow bark, dehydroepiandrosterone (DHEA), pregnenolone, ketone bodies, 5-hydroxytryptophan (5-HTP) and a racemic mixture of D,L phenylalanine (DLPA).

15. A method as in claim 14, wherein said ketone bodies comprise: β-hydroxybutyrate and acetoacetate, or their salts/precursor; and medium chain triglycerides or their C6 to C14 fatty acid analogs or precursors.

16. A composition for avoiding or treating chronic pain and/or age-related degeneration, said composition comprising an effective amount of: three agents selected from the group consisting of vinpocetine, huperzine A, magnesium, theanine, pyro-glutamic acid, dextromethorphan, and Vitex agnus-castus fruit; one agent selected from the group consisting of thiamin, niacin, pyridoxine, vanadium, and acetaminophen; and three agents selected from the group consisting of valerian spp, passiflora spp, magnolia spp, scutellaria spp, taurine, melatonin, GABA and black cohosh.

17. A composition as in claim 16 and further including an effective amount of an agent selected from the group consisting of white willow bark, dehydroepiandrosterone (DHEA), pregnenolone, ketone bodies, 5-hydroxytryptophan (5-HTP) and a racemic mixture of D,L phenylalanine (DLPA).

18. A composition as in claim 17, wherein said ketone bodies comprise: β-hydroxybutyrate and acetoacetate, or their salts/precursor; and medium chain triglycerides or their C6 to C14 fatty acid analogs or precursors.

19. A composition as in claim 16 comprising magnesium, vinpocetine, huperzine, theanine, pyridoxine, valerian spp, taurine, and magnolia spp.

20. A composition as in claim 19 and further comprising 5HTP and white willow bark.

21. A composition as in claim 19 and further comprising DHEA, melatonin, and Vitex agnus-castus fruit.

22. A composition as in claim 19 and further comprising medium chain triglycerides.

23. A composition as in claim 16 comprising vinpocetine, huperzine, magnesium, dextromethorphan, acetaminophen, taurine, black cohosh, and magnolia spp.

24. A composition as in claim 16 comprising magnesium, theanine, pyro-glutamic acid, thiamin, valerian spp, passiflora spp, magnolia spp, and white willow bark.

25. A composition as in claim 16 comprising magnesium, vinpocetine, huperzine, thiamin, niacin, pyridoxine, taurine, magnolia spp, melatonin, and MCT oil.

26. A composition as in claim 16 comprising theanine, magnesium, pyro-glutamic acid, thiamin, valerian spp, passiflora spp, magnolia spp, and DLPA.

27. A composition as in claim 16 comprising magnesium, Vitex agnus-castus fruit, acetaminophen, black cohosh, valerian spp, magnolia spp, melatonin, and 5 HTP.

28. A method for addressing chronic pain and/or degeneration, said method comprising orally or parenterally administering to the human for a effective period, an effective amount of a composition comprising: three agents selected from the group consisting of vinpocetine, huperzine A, magnesium, theanine, pyro-glutamic acid, dextromethorphan, and Vitex agnus-castus fruit; one agent selected from the group consisting of thiamin, niacin, pyridoxine, vanadium, and acetaminophen; and three agents selected from the group consisting of valerian spp, passiflora spp, magnolia spp, scutellaria spp, taurine, melatonin, GABA and black cohosh.

29. A method as in claim 28 wherein said composition further includes an agent selected from the group consisting of white willow bark, dehydroepiandrosterone (DHEA), pregnenolone, ketone bodies, 5-hydroxytryptophan (5-HTP) and a racemic mixture of D,L phenylalanine (DLPA).

30. A method as in claim 29, wherein said ketone bodies comprise: β-hydroxybutyrate and acetoacetate, or their salts/precursor; and medium chain triglycerides or their C6 to C14 fatty acid analogs or precursors.

31. A food, beverage, condiment, spice or salad dressing product designed for neurophysiologic health, said product comprising:
a food, beverage, condiment, spice or salad dressing base; and
a nutraceutical comprising an effective amount of: three agents selected from the group consisting of vinpocetine, huperzine A, magnesium, theanine, pyro-glutamic acid, dextromethorphan, and Vitex agnus-castus fruit; one agent selected from the group consisting of thiamin, niacin, pyridoxine, vanadium, and acetaminophen; and three agents selected from the group consisting of valerian spp, passiflora spp, magnolia spp, scutellaria spp, taurine, melatonin, GABA and black cohosh;
said nutraceutical present in an amount in excess of the amount of said nutraceutical naturally present in said food, beverage, condiment, spice or salad dressing base.

31. A product as in claim 31 wherein said base is a beverage base and said beverage is an alcoholic beverage.

32. A product as in claim 31 wherein said base is a food base and said food is selected from the group consisting of: vegetables and vegetable food products such as baked beans, tomato-based products like tomato paste, stewed tomatoes, spaghetti sauce, pizza sauce, vegetable oils, fruit and fruit food products such as jellies, jams and syrups, cereals, trail mix, cookies, pasta, flours including wheat, soy, oat, and potato flour, whey, chocolate, candy, tofu, bagels, baked goods such as bread, cakes, pizza dough and mixes for these, pancakes and waffles and mixes for these, soups, trail mix, nutritional bars, peanut butter, potato chips, corn chips, crackers, meats, and meat food products, such as lunch meats, hot dogs and sausage, milk food products, such as ice cream, yogurt, cheese and butter.

33. A product as in claim 31 wherein said base is a beverage base and said beverage is selected from the group consisting of: soft drinks, tea, coffee, milk, fruit juices, and sports drinks.

34. A product as in claim 31 wherein said base is a condiment or spice base and said condiment or spice is selected from the group consisting of: steak sauce, mustard, catsup, soy sauce, chili sauce, chip dips, salsa, pickles, horseradish, curry, chili powder, salt, pepper, vinegar, cinnamon, garlic and garlic powder, garlic oil, onion and onion powder and onion oil.

35. A product as in claim 31 wherein said nutraceutical further includes an effective amount of an agent selected from the group consisting of white willow bark, dehydroepiandrosterone (DHEA), pregnenolone, ketone bodies, 5-hydroxytryptophan (5-HTP) and a racemic mixture of D,L phenylalanine (DLPA).

36. A product as in claim 35 wherein said ketone bodies comprise: β-hydroxybutyrate and acetoacetate, or their salts/precursor; and medium chain triglycerides or their C6 to C14 fatty acid analogs or precursors.

37. A product as in claim 31 wherein said nutraceutical comprises magnesium, vinpocetine, huperzine, theanine, pyridoxine, valerian spp, taurine, and magnolia spp.

38. A product as in claim 37 wherein said nutraceutical further comprises 5HTP and white willow bark.

39. A product as in claim 37 wherein said nutraceutical further comprises DHEA, melatonin, and Vitex agnus-castus fruit.

40. A product as in claim 37 wherein said nutraceutical further comprises medium chain triglycerides.

41. A product as in claim 31 wherein said nutraceutical comprises vinpocetine, huperzine, magnesium, dextromethorphan, acetaminophen, taurine, black cohosh, and magnolia spp.

42. A product as in claim 31 wherein said nutraceutical comprises magnesium, theanine, pyro-glutamic acid, thiamin, valerian spp, passiflora spp, magnolia spp, and white willow bark.

43. A product as in claim 31 wherein said nutraceutical comprises magnesium, vinpocetine, huperzine, thiamin, niacin, pyridoxine, taurine, magnolia spp, melatonin, and MCT oil.

44. A product as in claim 31 wherein said nutraceutical comprises theanine, magnesium, pyro-glutamic acid, thiamin, valerian spp, passiflora spp, magnolia spp, and DLPA.

45. A product as in claim 31 wherein said nutraceutical comprises magnesium, Vitex agnus-castus fruit, acetaminophen, black cohosh, valerian spp, magnolia spp, melatonin, and 5 HTP.
